(19) United States
(12) Patent Application Publication
(10) Pub. No.: US 2006/0093633 A1
(43) Pub. Date: May 4, 2006

(54) Cosmetic and/or dermatological preparation comprising 2,3-dibenzylbutyrolactones

(76) Inventors: Franz Stab, Echem (DE); Rainer Wolber, Hamburg (DE); Thomas Blatt, Wedel (DE); Ludger Kolbe, Dohren (DE); Claudia Mundt, Bremen (DE); Stefan Galliant, Wedel (DE); Kirsten Venzke, Emden (DE); Karen Tom Dieck, Hamburg (DE); Ute Breitenbach, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/515,000

(22) PCT Filed: May 22, 2003
(86) PCT No.: PCT/EP03/05347

§ 371 Date: Sep. 26, 2005

(30) Foreign Application Priority Data
May 27, 2002 (DE) 10223486.8

(51) Int. Cl. A61K 8/97; A61K 8/73; A61K 36/28; A61K 31/365

(52) U.S. Cl. 424/401; 424/74; 424/70.13; 424/764; 514/471; 514/473

(57) ABSTRACT

Cosmetic and/or dermatological preparation containing one or more 2,3-dibenzylbutyrolactone derivatives and/or their glycosides besides optionally additional cosmetic and/or dermatological active ingredients, auxiliary agents, and additives.

The present invention relates to a cosmetic and/or dermatological preparation containing one or more 2,3-dibenzylbutyrolactone derivatives and/or their glycosides besides optionally further cosmetic and/or dermatological active ingredients, auxiliary agents, and additives.

The skin is the largest organ of humans. It fulfills a plurality of functions (for example, regulation of heat, sensory organ for the sense of touch and sensation of heat, barrier function, protection against drying).

The skin can be subdivided into three histologically definable layers:
Epidermis
Cutis, corium, dermis
Subcutis

The outer layer is formed by the epidermis. As a barrier layer, it forms the actual protective covering against the environment. With about one tenth of the total thickness, it is at the same time the thinnest layer of the skin. The epidermis is a stratified tissue, in which the outer layer, the Stratum corneum represents the most essential portion for the barrier function. In contact with the environment, it is subjected to wear and, therefore, undergoes a constant regeneration process. In this process, fine scales are continuously discharged toward the outside, and horny cell and lipid material is reproduced from the inside.

Below the epidermis is the cutis, which is also called corium or dermis. The main function of this mesodermal connective tissue consists in feeding the epidermis. It is formed by elastic, collagenic, and reticular fibers, and comprises a large number of plasma and mast cells.

The subcutis consists of a loose connective tissue with more or less numerous fat cells embedded therein. It serves as heat protection, mechanical cushioning, as well as a depot for nutrients and water.

The chronological aging of the skin is caused, for example, by endogenous, genetically determined factors. In the epidermis and dermis, aging causes, for example, the following structural damage and functional disorders, which may also fall under the term “senile xerosis,” namely:

a) dryness, roughness, and formation of dryness wrinkles;

b) itching; and

c) reduced refatting by sebaceous glands (for example, after washing).

Exogenous factors, such as UV light and chemical noxae, can have a cumulative effect, and accelerate or supplement, for example, endogenous aging processes. In the epidermis and dermis, exogenous factors cause in particular, for example, the following structural damages and functional disorders in the skin, which go beyond extent and quality of the damages in the case of chronological aging:

d) visible vascular dilations (teleangiectasis, cuperosis;

e) flaccidity and formation of wrinkles;

f) local hyperpigmentation, hypopigmentation, and abnormal pigmentation (for example, age spots);

g) increased susceptibility to mechanical stress (for example, cracking); and

h) decrease of the collagen content of the skin (for example, by reduced neosynthesis and/or increased decomposition), and/or disorders of the metabolism of glycosaminoglycane and/or elastin.

Besides skin aging processes, the external appearance of the skin as well as its function can be disturbed and damaged by an imbalance of the skin flora, increased resorption of toxic or allergenic substances, or an attack by skin-foreign microorganisms. As a consequence, these impairments may lead to toxic or allergenic skin reactions.

In the case of unclean skin, which is described the transitional state between the healthy normal skin and the abnormally changed acne skin, the skin produces increased amounts of sebum (seborrhea). This sebum serves as an ideal culture medium for numerous microorganisms, in particular Propionibacterium acnes and Pityrosporum species. The microorganisms decompose the sebum into glycerin and fatty acids, thereby stimulating the sebaceous glands to increased production, and attacking and destroying the follicle walls in the skin. This causes inflammation of the skin (pustules, nodes, cysts), which often heal only with scars, thereby permanently damaging the optical appearance of humans who suffer from unclean skin.

It is the object of cosmetic skin care to strengthen and restore the natural function of the skin as a barrier against environmental influences (for example, dirt, chemicals, microoganisms) and against the loss of endogenous substances (for example, water, natural fats, electrolytes). Thus, the use of cosmetics serves to slow down the skin aging process and to care for the natural skin flora.

In the art, a number of products have already been developed for the care of flaccid, in particular aged skin. They contain, for example, retinoids (vitamin A acid and/or its derivatives), or vitamin A and/or its derivatives. Their effect on structural damage, however, is limited in terms of extent. Moreover, great difficulties exist in the product development with respect to adequately stabilizing the active ingredients against oxidative decay. Beyond that, the use of vitamin A acid containing products often causes strong erythematous skin irritations. Retinoids can therefore be used only in low concentrations.

Likewise, a plurality of products has been developed in the art for the treatment and care of unclean skin. These products, however, involve the problem of an inadequate balancing of effect and side effect. Conventional products have either an excessively bactericidal and degreasing effect, whereby healthy skin flora is normally destroyed, or by which the skin is caused to dry out extremely, or the products are barely effective to ensure successful treatment within a reasonable time.

It was therefore an object of the present invention to eliminate or at least lessen the deficiencies of the art, and to develop a cosmetic and/or dermatological preparation, which is both prophylactically and therapeutically effective against all signs of skin aging and unclean skin.

Surprisingly, the object is accomplished by a cosmetic and/or dermatological preparation that contains one or more 2,3-dibenzylbutyrolactone derivatives and/or their glycosides besides optionally further cosmetic and/or dermatological active ingredients, auxiliary agents, and additives.

The preparation of the invention is highly effective against all aging signs of the skin. The development of wrinkles and creases, as well as age spots is clearly suppressed. Already existing wrinkles and creases disappear, flaccid skin becomes again tight, and assumes a fresh and youthful appearance. Existing age spots are allowed to disappear by the use of the preparation of the invention. Degenerated skin portions are regenerated, disturbed blood circulation is clearly lessened. Even itching and stress sensation of the skin ease noticeably. Moreover, the preparation of the invention is suited as an effective and yet mild agent for the prophylaxis and treatment of unclean skin and its abnormal protuberances in the form of acne.

The 2,3-dibenzylbutyrolactone derivatives and/or their glycosides as used by the present invention are derived from the 2,3-dibenzylbutyrolactone as likewise used by the present invention, which is characterized by the following structure:

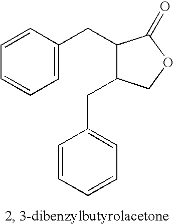

In accordance with the invention, use is made of 2,3-dibenzylbutyrolactone, 2,3-dybenzylbutyrolactone derivatives, and/or their glycosides in all their stereoisomeric forms, which can be present both as racemate and in the form of a pure enantiomorph, as well as in racemic mixtures with different enantiomorphic constituents. In accordance with the invention, the formulation comprises 2,3-dibenzylbutyrolatone derivatives and/or their glycosides as well 2,3-dibenzylbutyrolactone.

In accordance with the invention, the cosmetic and/or dermatological preparation of the invention is preferably present in the form of an emulsion. The preparations for the purposes of the present invention may preferably contain besides one or more oil phases, in addition one ore more water phases, and be present, for example, in the form of W/O (water-in-oil), W/S (water-in-silicone oil), O/W (oil-in-water), S/W (silicone oil-in-water) emulsions. Furthermore, in accordance with the invention they may advantageously be likewise present in so-called multiple emulsions, such as, for example, W/O/W or O/W/O emulsions. Preferably, such formulations may also be a microemulsion (for example, a PIT emulsion), a solid emulsion (i.e., an emulsion that is stabilized by solids, for example, a Pickering emulsion), a sprayable emulsion or a hydrodispersion. Furthermore, the preparations for the purposes of the invention can also be almost waterfree (water content less than 5% by weight based on the total weight of the preparation). Likewise, aqueous solutions are advantageous in accordance with the invention. According to the invention, the preparations are advantageously also present in the form of lipodispersions, gels, solid sticks, or aerosols.

Preparations for the purposes of the present invention may be present, for example, in the form of a cream, a lotion, a cosmetic milk, or a mousse-cream from an aerosol container.

It is also possible and advantageous for the purposes of the present invention to incorporate the 2,3-dibenzylbutyrolactone derivatives of the invention and/or their glycosides into aqueous systems or surfactant preparations for cleansing the skin.

In accordance with the invention, the concentration of one or more 2,3-dibenzylbutyrolactone derivatives and/or their glycosides advantageously ranges from 0.001% to 10% by weight, preferably 0.05% to 5% by weight, and very preferably from 0.01% to 2% by weight, each based on the total weight of the preparation.

The 2,3-dibenzylbutyrolactone derivatives of the invention and/or their glycosides can be added to the preparation of the invention advantageously in the form of plant extracts. Especially satisfactory in this connection are aqueous-alcoholic extracts from plants. However, according to the invention, it is also advantageous to include in the formulation of the preparations, extracts and distillates obtained by other forms of extraction and methods, for example, extracts obtained with the use of carbon dioxide as extraction agent, as well as water vapor distillates.

In this connection, it is especially advantageous in accordance with the invention to use plant extracts from Arctium lappa L. (greater burdock) and/or Steganotaenia araliacea (carrot tree).

In accordance with the invention, it is preferred to use as 2,3-dibenzylbutyrolactone derivatives and/or their glycosides, arctiin, arctigenin, prestegane B, tracheloside and/or trachelogenin.

Especially preferred in accordance with the invention are the derivatives of the following stereochemical structure: 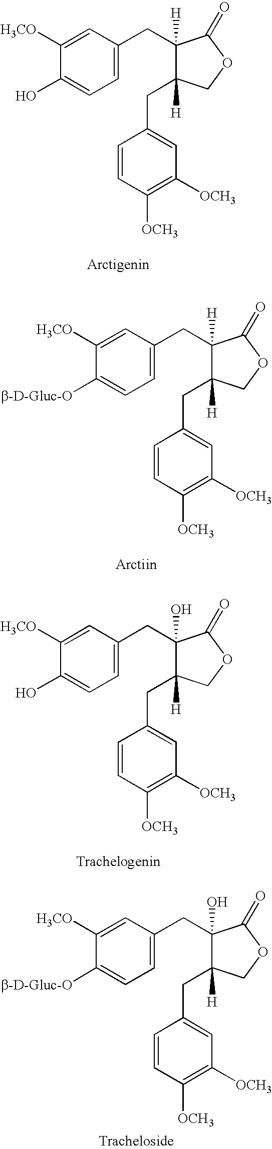 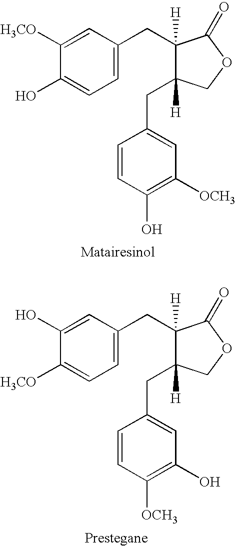
Especially preferred in accordance with the invention are arctiin and prestegane B.

It is easy to incorporate the 2,3-dibenzylbutyrolactone derivatives of the invention and/or their glycosides not only into customary cosmetic and/or dermatological preparations, such as light-protective preparations, skin care preparations, antiwrinkle preparations, but also into other preparations, for example, pharmaceutical preparations.

Cosmetic and/or dermatological preparations normally contain a plurality of auxiliary agents and active ingredients, which are also possible and advantageous to use in the preparations of the invention.

In accordance with the invention, it is possible to use customary antioxidants in the preparations that contain 2,3-dibenzylbutyrolactone derivatives and/or their glycosides.

Advantageously, the antioxodants are selected from the group consisting of amino acids (for example, glycine, histidine, tyrosine, tryptophan) and their derivatives; imidazoles (for example, urocanic acid) and their derivatives; peptides, such as D,L-carnosine, D-carnosine, L-carnosine, and their derivatives (for example, anserine); carotenoids, carotene (for example, alpha-carotene, beta-carotene, lycopene) and their derivatives; aurothioglucose, propylethiouracil and other thiols (for example, thioredoxin, glutathione, cysteine, cystine, cystamine, and their glycosyl-, N-acetyl-, methyl-, ethyl-, propyl-, amyl-, butyl- and lauryl-, palmitoyl-, oleyl-, γ-linoleyl-, cholesteryl-, and glyceryl esters), as well as their salts, dilaurylthiodipropionate, distearylhiodipropionate, thiodipropionic acid and their derivatives (ester, ether, peptides, lipids, nucleotides, nucleosides and salts), as well as metal chelators (for example, α-hydroxy fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA and their derivatives; unsaturated fatty acids and their derivatives (for example, γ-linolenic acid, linoleic acid, oleic acid, folic acid and their derivatives; alanine diacetic acid, flavonoids, polyphenoles, catechins, vitamin C and derivatives (for example, ascorbyl palmitate, Mg-ascorbyl phosphate, ascorbyl acetate); tocopherols and derivatives (for example, vitamin E acetate); as well as coniferyl benzoate of the benzoic resin; rutinic acid and its derivatives; ferulic acid and its derivatives; butylhydroxytoluol; butylhydroxyanisol; nordihydroguaiaic resin acid; nordihydroguaiaretic acid; trihydroxybutyrophenone; uric acid; and its derivatives, mannose and its derivatives; zinc and its derivatives (for examle, ZnO, ZnSO₄); selenium and its derivatives (for example, selenium methionine); stilbene and its derivatives (for example, stilbene oxide, trans-stilbene oxide); and the derivatives (salts, esters, ethers, sugar, nucleotides, nucelosides, peptides, and lipids) of these mentioned active substances, which are suited according to the invention.

The quantity of the antioxidants (one or more compounds) in the preparations is preferably from 0.001 to 10% by weight, very preferably 0.025 to 2.0% by weight, in particular 0.05 to 10% by weight based on the total weight of the preparation.

If vitamin A or vitamin A derivatives, or carotenes or their derivatives represent the antioxidant or antioxidants, it will be advantageous to select their respective concentrations in the range from 0.001 to 10% by weight based on the total weight of the formulation.

If vitamin E and/or its derivatives represent the antioxidant or antioxidants, it will be advantageous to select their respective concentrations from the range of 0.001 to 10% by weight based on the total weight of the formulation.

Further advantageous active ingredients for the purposes of the present invention are natural ingredients and/or their derivatives, such as, for example, alpha-lipoic acid, phytoene, D-biotin, coenzyme Q10, alpha-glucosylrutin, carnitine, carnosine, natural and/or synthetic isoflavonoids, creatine, creatinine, taurine, and/or beta-alanine, which can be used preferably in a concentration of 0.001 to 10% by weight based on the total weight of the preparation. It is also possible and advantageous to add to the preparations of the invention, hop extract or hop-malt extract, and/or soybean extracts, and/or clover extracts in a concentration of 0.001 to 10% by weight based on the total weight of the preparation.

Formulations of the invention, which contain, for example, known antiwrinkle agents, such as flavone glycosides (in particular alpha-glycosyl rutin), coenzyme Q 10, vitamin E and/or derivatives, and the like, are suited in a very advantageous manner for the prophylaxis and treatment of cosmetic or dermatological skin changes, as occur, for example, in skin aging (such as, for example, dryness, roughness, and formation of dryness wrinkles, itching, reduced refatting (for example, after washing), visible vascular dilations (teleangiectases, cuperosis), flaccidity and formation of wrinkles and lines, local hyperpigmentation, hypopigmentation, abnormal pigmentation (for example, age spots), increased susceptibility to mechanical stress (for example, cracking), and the like. Furthermore, they are advantageously suited for the treatment and prophylaxis of the clinical appearance of dry and rough skin, as well as for the treatment and prophylaxis of the symptoms of UV-light-induced skin aging (photoaging).

Naturally, it is known to the person of skill in the art that in most cases, exacting cosmetic preparations are not possible without the customary auxiliary agents and additives. The cosmetic preparations according to the invention can therefore contain cosmetic auxiliary agents as are customarily used in such preparations, for example, emulsifiers, preservatives, preservation aids, bactericides, perfumes, UV-light protection filters, skin-bleaching agents, selftanners, repellants, antifoaming agents, dyes, pigments with a coloring action, thickeners, moisturizers and/or humectants, fillers that improve dermal sensation, fats, oils, waxes, or other customary ingredients of a cosmetic or dermatological formulation, such as alcohols, polyols, polymers, foam stabililzers, electrolytes, organic solvents, or silicone derivatives.

Medicated topical compositions for the purposes of the present invention normally contain one or more medicaments in active concentrations. For the sake of simplicity, the legal provisions of the Federal Republic of Germany (for example, statutory regulation for cosmetics, food and medicament law) are herewith incorporated by reference for purposes of making a clear distinction between cosmetic and medicinal application and corresponding products.

Advantageously, preparations of the present invention may additionally contain substances, which absorb UW radiation in the UVB range, where the total amount of the filter substances is, for example, from 0.1% to 30% by weight, preferably 0.5% to 10% by weight, in particular 1.0% to 6.0% by weight based on the total weight of the preparations for making available cosmetic preparations, which protect hair and skin against the entire range of the ultraviolet radiation. They can also be used as sunscreen agents for the hair.

When the preparations of the present invention contain UVB filter substances, same may be oil-soluble or water-soluble. In accordance with the invention, advantageous oil-soluble UVB filters include, for example:

derivatives of 3-benzylidene camphor, preferably 3-(4-methylbenzylidene) camphor, 3-benzylidene camphor;

derivatives of 4-aminobenzoic acid, preferably 2-(ethylhexyl)4-dimethylamino-benzoate, amyl 4-(dimethylamino)-benzoate;

esters of cinnamic acid, preferably (2-ethylhexyl)4-methoxy cinnamate, isopentyl 4-methoxy cinnamate;

esters of salicylic acid, preferably (2-ethylhexyl) salicylicate, (4-isopropylbenzyl) salicylicate, homomenthyl salicylicate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate; and

2,4,6-trianilino-(p-carbo-2′-ethyl-1′-hexyloxy)-1,3,5-triazine.

Advantageous water-soluble UVB filters are, for example:

salts of 2-phenylbenzimidazole-5-sulfonic acid, such as its sodium-, potassium-, or its triethanol ammonium salt, as well as the sulfonic acid itself;

sulfonic acid derivatives of benzophenones, preferably, 2-hydroxy-4-methoxybenopenone-5-sulfonic acid and its salts;

sulfonic acid derivatives of 3-benzylidene camphor, such as, for example, 4-(2-oxo-3-bornylidene-methyl)benzenesulfonic acid, 2-methyl-5-(2-oxo-3-bornylidenemethyl)sulfonic acid and its salts, as well as the 1,4-di(2-oxo-10-sulfo-3-bornylidene)benzene and salts thereof (the corresponding 10-sulfato compounds, for example, the corresponding sodium-, potassium-, or triethanolammonium salt), also referred to as benzene-1,4-di(2-oxo-3-bornylidenemethyl-10-sulfonic acid;

hydroxybenzophenone derivatives, such as, for example, 2-(4-diethylamino-2-hydroxybenzoyl)benzoic acid hexyl ester, which is available from BASF under the trade name Uvinul® A Plus;

benzoxazol derivatives, such as, for example, 2,4-bis-[5-1(dimethylpropyl)benzoxazol-2-yl-4 (phenyl)-imino]-6-(2-ethylhexyl)-imino-1,3,5-triazine, (CAS No. 288254-16-0), which is available, for example, under the trade name UVASorb® K2A from 3V Sigma.

Naturally, the list of the referenced UVB filters, which may be used together with the combinations of active ingredients according to the invention, is not intended to be limiting.

It can also be of advantage to use UVA filters, which are normally present in cosmetic preparations. These substances are preferably derivatives of dibenzoylmethane, in particular 1-(4′-tert-butylphenyl)-3-(4′-methoxyphenyl)-propane-1,3-dione and 1-phenyl-3-(4′-isopropylphenyl)propane-1,3-dione. Furthermore, advantageous UVA filters are from the group of the triazines, for example 2,4-bis-{[4-(2-ethyl-hexyloxy)-2-hydroxy]-phenyl}-6-(4-methoxyphenyl)-1,3,5 triazine (trade name Tinosorb® S), as well as from the group of the triazoles, such as, for example, 2,2′-methylene-bis-[6-2H-benzotriazole-2-yl]-4-(1,1,3,3-tetramethylbutyl)-phenol (trade name Tinosorb® M). An advantageous water-soluble UVA filter is 2′-bis-(1,4-phenylene)-1H-benzimidazole-4,6-disulfonic acid sodium salt (trade name Neo Heliopan AP®).

The amounts used can be the same as the amounts for the UVB combination.

Advantageously, cosmetic and/or dermatological preparations in accordance with the invention contain in addition inorganic pigments based on metal oxides and/or other metal compounds that are difficult to dissolve in water or insoluble, in particular the oxides of titanium (TiO₂), zinc (ZnO), iron (for example, Fe₂O₃), zirconium (ZrO₂), silicon (SiO₂), manganese (for example, MnO) , aluminum (Al₂O₃), cerium (for example (Ce₂O₃), mixed oxides of the corresponding metals, as well as mixtures of such oxides. Especially preferred are pigments on the basis of TiO₂.

While not required, it will be especially advantageous for the purposes of the present invention, when the inorganic pigments are present in hydrophobic form, i.e., they are treated for water-repellency on their surface. This surface treatment may consist in that the pigments are provided with a thin, hydrophobic layer by a method known per se.

One of such methods consists, for example, in producing the hydrophobic surface layer by a reaction according to
n TiO₂+m(RO₃)Si—R′→n TiO₂ (superficial),
where n and m are stoichiometric parameters that are to be inserted as desired, and R and R′ are the desired organic radicals. For example, pigments that are made hydrophobic analogously to DE-OS 33 14 742 are of advantage.

Advantageous TiO₂ pigments are commercially available under the trade names MT 100T from TAYCA, furthermore M 160 from Kemira, as well as T 805 from Degussa.

Preparations according to the invention can also contain anionic, nonanionic, and/or amphoteric surfactants, in particular when crystalline or microcrystalline solids, for example, inorganic micropigments are to be included in the preparations of the present invention. Surfactants are amphophilic substances, which are capable of dissolving organic, nonpolar substances in water.

The hydrophilic moieties of a surfactant molecule are in most cases polar functional groups, for example —COO⁻, —OSO₃²⁻, —SO₃⁻, whereas the hydrophobic parts normally represent nonpolar hydrocarbon residues. In general, surfactants are classified according to the type and charge of the hydrophilic moiety of the molecule. In this connection, it is possible to differentiate between four groups:
anionic surfactants;
cationic surfactants;
amphoteric surfactants; and
nonionic surfactants.

As functional groups, anionic surfactants normally include carboxylate-, sulfate-, and sulfonate groups. In an aqueous solution, they form negatively charged, organic ions in the acidic or neutral environment. Cationic surfactants are characterized nearly exclusively by the presence of a quaternary ammonium group. In an aqueous solution, they form positively charged, organic ions in the acidic or neutral environment. Amphoteric surfactants contain both anionic and cationic groups, and act accordingly in an aqueous solution as anionic or cationic surfactants depending on the pH value. In a strongly acidic environment, they possess a positive charge, and in an alkaline environment a negative charge. In the neutral pH range, however, they are zwitterionic, as is demonstrated in the following example:

| | | |
|---|---|---|
| RNH₂⁺₂CH₂COOHX⁻ | (at pH = 2) | X⁻ = any desired |
| | | anion, e.g., Cl⁻ |
| RNH₂⁺CH₂CH₂COO⁻ | (at pH = 7) | |
| RNHCH₂CH₂COO⁻B⁺ | (at pH = 12) | B⁺ = any desired |
| | | cation, e.g., |
| | | Na⁺ |

Typical of nonionic surfactants are polyether chains. Nonionic surfactants form no ions in an aqueous medium.

### A. Anionic Surfactants

Anionic surfactants that can be advantageously used, include: acylamino acids (and salts thereof), such as:

1. Acylglutamate, for example, sodium acyl glutamate, di-TEA-palmitoyl asparate, and sodium caprylic/capric glutamate;

2. Acylpeptides, for example, palmitoyl-hydrolyzed milk protein, sodium cocoyl-hydrolyzed soybean protein, and sodium/potassium cocoyl-hydrolyzed collagen;

3. Sarcosinates, for example, myristoyl sarcosinate, TEA-lauroyl sarcosinate, sodium lauroyl sarcosinate, and sodium cocoyl sarcosinate;

4. Taurates, for example, sodium lauroyl taurate and sodium methylcocoyl taurate;

5. Acyl lactylates, lauroyl lactylate, caproyl lactylate; and

6. Alaninates.

Carboxylic acids and derivatives, such as:

1. Carboxylic acids, for example, lauric acid, aluminum stearate, magnesium alkanolate, and zinc undecylenate;

2. Ester carboxylic acids, for example, calcium stearoyl lactyllate, laureth-6 citrate, and sodium PEG-4 lauramide carboxylate; and

3. Ether carboxylic acids, for example, sodium laureth-13 carboxylate, and sodium PEG-6 cocoamide carboxylate.

Esters of phosphoric acid and salts, such as, for example, DEA-oleth-10-phosphate and dilaureth-4 phosphates.

Sufonic acids and salts, such as:

1. Acyl isethionate, for example, sodium-ammoniumcocoyl isethionate;

2. Alkyaryl sulfonates;

3. Alkyl sulfonates, for example, sodium cocosmonoglyceride sulfate, sodium C₁₂₋₁₄olefin sulfonate, sodium lauryl sulfoacetate, and magnesium PEG-3 cocamide sulfate;

4. Sulfosuccinates, for example, dioctyl sodium sulfosuccinate, disodium laureth sulfosuccinate, disodium lauryl sulfosuccinate, and disodium undecylenamido-MEA-sulfosuccinate; as well as

Esters of sulfuric acid, such as:

1. Alkyl ether sulfonates, for example, sodium, ammonium, magnesium, MIPA, TIPA, laureth sulfate, sodium myreth sulfate, and sodium C₁₂₋₁₃pareth sulfate; and

2. Alkyl sulfates, for example, sodium, ammonium, and TEA-lauryl sulfate.

### B. Cationic Surfactants

Cationic surfactants that can be advantageously used, include:

1. Alkylamines,

2. Alkylimidazoles,

3. Ethoxylated amines,

4. Quaternary surfactants, and

5. Esterquats.

Quaternary surfactants contain at least one nitrogen atom, which is covalently bonded with 4 alkyl- or aryl groups. Irrespective of the pH value, this leads to a positive charge. Advantageous are alkylbetaine, alkylamidopropylbetaine, and alkyl-amidopropylhydroxysulfaine. The cat ionic surfactants that are used in accordance with the invention, can also be selected from the group of quaternary ammonium compounds, in particular benzyltrialkyl ammoniumchlorides or bromides, such as, for example, benzyldimethylstearyl ammonium chloride, furthermore alkyltrialkyl ammonium salts, for example, cetyltrimethyl ammonium chloride or bromide, alkyidimethylhydroxyethyl ammonium chloride or bromide, dialkyldimethyl ammonium chloride or bromide, alkylamidethyltrimethyl ammonium ether sulfates, alkylpyridinium salts, for example, lauryl or cetyl pyrimidinium chloride, imidazoline derivatives and compounds having cationic character, such as amine oxides, for example, alkyldimethylamine oxide or alkylaminoethyl dimethylamine oxide. The use of cetyltrimethyl ammonium salts is particularly advantageous.

### C. Amphoteric Surfactants

Amphoteric surfactants that can be advantageously used, include:

1. Acyl-/dialkylethyl endiamine, for example, sodium acyl amphoacetate, disodiumacyl amphodipropionate, disodium alkyl amphodiacetate, sodium acylamphohydroxy propylsulfonate, disodium acyl amphodiacetate, and sodium acyl amphopropionate;

2. N-alkylamino acids, for example, aminopropyl-alkylglutamide, alkylaminopropionic acid, sodium-alkylimidodipropionate and lauroamphocarboxyglycinate.

### D. Nonionische Surfactants

Nonionic surfactants, which can be advantageously used, include:

1. Alcohols;

2. Alkanolamides, such as MEA/DEA/MIPA cocoamides;

3. Amine oxides, such as cocoamidopropylamine oxide;

4. Esters, which result from the esterification of carboxylic acids with ethylene oxide, glycerin, sorbitan, or other alcohols;

5. Ethers, for example, ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE-ethers, and alkyl polyglycosides, such as lauryl glucoside, decyl glycoside and coco glycoside;

6. Sucrose esters, sucrose ethers;

7. Polyglycerol esters, diglycerol esters, monoglycerol esters; and

8. Methylglucose esters, esters of hydroxy acids.

It is also advantageous to use a combination of anionic and/or amphoteric surfactants with one or more nonionic surfactants.

In the preparations of the invention, the surface-active substance may be present in a concentration between 1 and 30% by weight based on the total weight of the preparations.

The cosmetic or dermatological preparations of the invention can be composed in the usual manner and be used for treating, caring for, and cleansing the skin and/or hair, and serve as makeup product in decorative cosmetics. Accordingly, depending on their composition, they can be used, for example, as skin protection cream, cleansing milk, sunscreen lotion, nourishing cream, day cream or night cream, etc. In some instances, it is possible and advantageous to use the preparations of the invention as base for pharmaceutical formulations. The preparations of the invention contain active ingredients of the invention, for example, in a range from 0.001 to 30% by weight, preferably, 0.01 to 10% by weight, in particular however 0.1 to 5% by weight, each based on the total weight of the preparations.

In accordance with the invention, the cosmetic and dermatological preparations for protecting hair against UV radiation include, for example, shampooing agents; preparations, which are used for rinsing the hair before or after shampooing, before or after a permanent wave treatment, before or after coloring and decoloring the hair; preparations for blow drying or setting hair; preparations for coloring and decoloring; a hair-styling and treating lotion; a hair spray, or permanent wave agent.

The lipid phase of the cosmetic or dermatological preparations according to the invention may advantageouly be selected from the following group of substances:

mineral oils, mineral waxes;

oils, such as triglycerides of capric or capryllic acids, furthermore natural oils, such as for example, castor oil;

fats, waxes, and other natural and synthetic lipoids, preferably esters of fatty acids with alcohols having a low carbon number, e.g., with isopropanol, propylene glycol or glycerol, or esters of fatty alcohols with alkanoic acids of a low carbon number or with fatty acids;

alkylbenzoates; and

silicone oils, such as dimethylpolysiloxanes, diethylpolysiloxanes, diphenylpolysiloxanes, as well as mixed forms thereof.

Advantageously, the oil phase of the preparations of the present invention may further be selected from the groups of the esters of saturated and/or unsaturated, branched and/or unbranched alkane carboxylic acids having a chain length from 3 to 30 carbon atoms and saturated and/or unsaturated, branched and/or unbranched alkohols having a chain length from 3 to 30 carbon atoms; from the group of the esters of aromatic carboxylic acids and saturated and/or unsaturated, branched and/or unbranched alkohols having a chain length from 3 to 30 carbon atoms. Such ester oils may advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethyl hexyl palmitate, 2-ethylhexyl laurate, 2-hexyldecyl stearate, 2-octyldodecyl palmitate, oleyl oleate, oley lerucate, erucyl oleate, erucyl erucate, as well as synthetic, semisynthetic, and natural mixtures of such esters, for example, jojoba oil.

Furthermore, the oil phase may advantageously be selected from the group of the branched and unbranched hydrocarbons and hydrocarbon waxes, the silicone oils, the dialkyl ethers, the group of the saturated or unsaturated, branched or unbranched alcohols, as well as the fatty acid triglycerides, namely the triglycerol esters of saturated and/or unsaturated, branched and/or unbranched alkane carboxylic acids having a chain length from 8 to 24, in particular 12 to 18 carbon atoms. The fatty acid triglycerides may adantageously be selected, for example, from the group of the synthetic, semisynthetic and natural oils, for example, olive oil, sunflower seed oil, soybean oil, peanut oil, rape seed oil, almond oil, palm oil, coconut oil, palm kernel oil, and the like.

Any mixtures of such oil and wax components can also be used advantageously for the purposes of the present invention. In some instances, it may also be advantageous to use waxes, for example, cetyl palmitate, as the only lipid component of the oil phase.

Advantageously, the oil phase is selected from the group of 2-ethylhexylstearate, octyldodecanol, isotridecyl isononanoate, isoeicosane, 2-ethylhexylcocoate, C₁₂₋₁₅-alkylbenzoate, triglyceride of the caprylic-capric acid, dicaprylylether.

Especially advantageous are mixtures of C₁₂₋₁₅ alkyl benzoate and 2-ethylhexyl isostearate, mixtures of C₁₂₋₁₅ alkyl benzoate and isotridecyl isononanoate, as well as mixtures of C₁₂₋₁₅ alkyl benzoate, 2-ethylhexyl-isostearate and isotridecyl isononanoate.

Of the hydrocarbons, paraffin oil, squalane and squalene are to be used with advantage for the purposes of the present invention.

Advantageously, the oil phase can also advantageously include a content of cyclic or linear silicone oils or completely consist of such oils, although it is preferred to use an additional content of other oil phase components apart from the silicone oil or silicone oils. Such silicones or silicone oils may be present as monomers, which are normally characterized by structural elements, as follows:

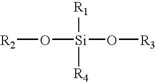

Linear silicones that are to be advantageously used according to the invention with a plurality of siloxyl units, are generally characterized by structural elements, as follows: 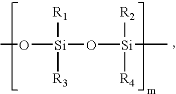
where the silicon atoms with the same or different alkyl radicals and/or aryl radicals may be substituted. They are here generally shown by the radicals R₁—R₄ (which means that the number of the different radicals is not necessarily limited to as many as 4), and m may assume values from 2 to 200,000.

Cyclical silicones that are to be advantageously used in accordance with the invention, are generally characterized by structural elements, as follows: 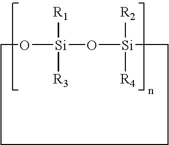
where the silicon atoms with the same or different alkyl radicals and/or aryl radicals, which are in general shown by the radicals R₁—R₄, may be substituted (which means that the number of the different radicals is not necessarily limited to as many as 4). n may assume values from 3/2 to 20. Broken values for n take into account that odd numbers of siloxyl groups may be present in the cyclic structure.

Advantageously, cyclomethicone (for example, decamethylcyclopentasiloxane) is used as silicone oil in accordance with the invention. However, other silicone oils can also be used advantageously in the meaning of the present invention, for example, undecamethylcyclotrisiloxane, polydimethylsiloxane, poly(methylphenylsiloxane), cetyldimethicone, and behenoxydimethicone.

Mixtures of cyclomethicone and isotridecyl isononanoate, as well as such of cyclomethicone and 2-ethyl hexyl isostearate are also advantageous.

However, it is also advantageous to select silicone oils of a constitution similar to that of the above-described compounds, whose organic side chains are derivatized, for example, polyethoxylated and/or polypropoxylated. These include, for example, polysiloxanepolyalkyl-polyether-copolymers, such as the cetyl-dimethicone-copolyol, cetyl-dimethiconecopolyol (and) polyglyceryl-4-isostearate (and) hexyl laurate).

Especially advantageous are also mixtures of cyclomethicone and isotridecyl isononanoate, of cyclomethicone and 2-ethyl hexyl isostearate.

Advantageously, the aqueous phase of the preparations according to the invention include in some instances low-carbon alkohols, diols or polyols, as well as ethers thereof, preferably ethanol, isopropanol, propylene glycol, glycerol, ethylene glycol, ethylene glycol monoethyl or monobutyl ether, propylene glycol monomethyl, monoethyl, or monobutyl ether, diethylene glycolmonomethyl or monoethyl ether und analogous products, furthermore alcohols of a low carbon number, for example, ethanol isopropanol, 1,2-propane-diol, glycerol as well as in particular, one or more thickeners, which can advantageously be selected from the group of silicon dioxide and aluminum silicates.

Preparations of the present invention advantageously contain in particular one or more hydrocolloids. These hydrocolloids may advantageously be selected from the group of the gums, polysaccharides, cellulose derivatives, layer silicates, polyacrylates and/or other polymers.

Preparations of the invention that are present as hydrogels, contain one or more hydrocolloids. These hydrocolloids can advantageously be selected from the aforesaid group.

Gums include plant or tree saps, which cure in the air and form resins, or extracts from water plants. From this group, it is advantageous to select, for the purposes of-the present invention, for example, gum arabicum, carob meal, tragacanth, Karaya, guar gum, pectin, gellan gum, carrageen, agar, algin, chondrus, xanthan gum.

Furthermore, it is advantageous to use derived gums, such as, for example, hydroxypropyl guar (Jaguar® HP 8).

Polysaccharides and derivatives thereof include, for example, hyaluronic acid, chitin, and chitosan, chondroitin sulfate, starch, and starch derivatives.

Cellulose derivatives include, for example, methylcellulose, carboxymethyl cellulose, hydroxyethyl cellulose, and hydroxypropyl methylcellulose.

Layer silicates include naturally occurring and synthetic clays, such as, for example, montmorillonite, bentonite, hectorite, laponite, magnesium-aluminum silicates, such as Veegum®. They may be used as such or in modified form, such as, for example, stearyl alkonium hectorite.

Furthermore, it is also advantageous to use silicic-acid gels.

Polyacrylates include, for example, Carbopol™ types from Goodrich (Carbopol™ 980, 981, 1382, 5984, 2984, EDT 2001, EDT 2020, EDT 2050, Ultrez 10, or Pemulen TR1 & TR2).

Polymers include, for example, polyacrylamides (Seppigel 305), polyvinyl alcohols, PVP, PVP/VA copolymers, and polyglycols.

Preparations according to the invention, which are present as emulsions, include one more emulsifiers. Advantageously, these emulsifiers can be selected from the group of the nonionic, anionic, cationic, or amphoteric emulsifiers.

Nonionic emuslifiers include:

a) partial fatty acid esters and fatty acid esters of polyvalent alcohols and their ethoxylated derivatives (for example, glyceryl monostearates, sorbitan stearates, glyceryl stearyl citrates, sucrose stearates);

b) ethoxylated fatty alcohols and fatty acids;

c) ethoxylated fatty amines, fatty acid amides, fatty alkanolamides;

d) alkylphenolpolyglycolether (for example, Triton X); and

e) sugar derivatives (ester and/or ether of glucose, saccharose, and other sugars; for example, alkyl polyglycosides, such as polyglyceryl-3-methylglucose distearate, and methylglucose sequistearate).

Anionic emulsifiers include:

a) soaps (for example, sodium stearate);

b) fatty alcohol sulfates;

c) mono-, di- and trialkyl phosphoric acid esters and their ethoxylates.

Cationic emulsifiers include:

a) quaternary ammonium compounds with a long-chain aliphatic residue, for example, distearyl diammonium chlorides.

Among the amphoteric emsulsifiers are:

a) alkyl aminino alkane carboxylic acids;

b) betaines, sulfobetaines;

c) imidazolin derivatives.

Furthermore, there are naturally occurring emulsifiers, which include beeswax, wool wax, lecithin, and sterols.

O/W emulsifiers can advantageously be selected, for example, from the group of polyethoxylated or polypropoxylated or polyethoxylated and polypropoxylated products, for example:
fatty alcohol ethoxylates;
ethoxylated wool wax alcohols;
polyethylene glycol ethers of the general formula R—O—(—CH₂—CH₂—O)ₙ—R′;
fatty acid ethoxylates of the general formula R—COO—(—CH₂—CH₂—O—)ₙ—H;
etherified fatty acid ethoxylates of the general formula R—COO—(—CH₂—CH₂—O—)ₙ—R′;
esterified fatty acid ethoxylates of the general formula R—COO—(—CH₂—CH₂—O—)ₙ—C(O)—R′;
fatty acid esters of polyethylene glycolglycerin;
ethoxylated sorbitanester;
cholesterol ethoxylates;
ethoxylated triglycerides;
alkyl ether carboxylic acids of the general formula R—O—(—CH₂—CH₂—O—)—CH₂—COOH nd, where n is a number from 5 to 30;
fatty acid ester of polyoxyethylene sorbitol;
alkylether sulfates of the general formula R—O—(—CH₂—CH₂—O—)ₙ—SO₃—H;
fatty alcohol propoxylates of the general formula R—O—(—CH₂—CH(CH₃)—O—)ₙ—H;
polypropylene glycol ethers of the general formula R—O—(—CH₂—CH(CH₃)—O—)ₙ—R′;
propoxylated wool wax alcohols;
etherified fatty acid propoxylates R—COO—(—CH₂—CH(CH₃)—O—)ₙ—R′;
esterified fatty acid propoxylates of the general formula R—COO—(—CH2—CH(CH3)—O—)ₙC(O)—R′;
fatty acid propoxylates of the general formula R—COO—(—CH₂—CH(CH₃)—O—)ₙ—H;
fatty acid esters of polypropylene glycolglycerin;
propoxylated sorbitan esters;
cholesterol propoxylates;
propoxylated triglycerides;
alkyl ether carboxylic acids of the general formula R—O—(—CH₂—CH(CH₃)—O—)ₙ—CH₂—COOH;
alkylether sulfates or the parent acids of these sulfates of the general formula R—O—(—CH₂—CH(CH₃)—O—)ₙ—SO₃—H;
fatty alcohol ethoxylates/propoxylates of the general formula R—O—Xₙ—Yₘ—H;
polypropylene glycol ethers of the general formula R—O—Xₙ—Yₘ—R′;
etherified fatty acid propoxylates of the general formula R—COO—Xₙ—Yₘ—R′;
fatty acid ethoxylates/propoxylates of the general formula R—COO—Xₙ—Yₘ—H.

In accordance with the invention, it is especially advantageous to select the polyethoxylated or polypropoxylated or polyethoxylated and polypropoxylated O/W emulsifiers from the group of the substances with HLB values from 11 to 18, very advantageously with HLB values from 14.5 to 15.5, provided the O/W emulsifiers have saturated radicals R and R′. If the O/W emulsifiers include unsaturated radicals R and/or R′, or if isoalkyl derivatives are present, the preferred HLB value of such emulsifiers can also be lower or higher.

It is advantageous to select the fatty alcohol ethoxylates from the group of the ethoxylated stearyl alcohols, cetyl alcohols, cetyl stearyl alcohols (cetearyl alcohols). Especially preferred are:
Polyethylene glycol(13)stearyl ether (steareth-13),
polyethylene glycol(14)stearyl ether (steareth-14),
polyethylene glycol(15)stearyl ether (steareth-15),
polyethylene glycol(16)stearyl ether (steareth-16),
polyethylene glycol(17)stearyl ether (steareth-17),
polyethylene glycol(18)stearyl ether (steareth-18),
polyethylene glycol(19)stearyl ether (steareth-19),
polyethylene glycol(20)stearyl ether (steareth-20);
Polyethylene glycol(12)isostearyl ether (isosteareth-12),
polyethylene glycol(13)isostearyl ether (isosteareth-13),
polyethylene glycol(14)isostearyl ether (isosteareth-14),
polyethylene glycol(15)isostearyl ether (isosteareth-15),
polyethylene glycol(16)isostearyl ether (isosteareth-16);
polyethylene glycol(17)isostearyl ether (isosteareth-17),
polyethylene glycol(18)isostearyl ether (isosteareth-18),
polyethylene glycol(19)isostearyl ether (isosteareth-19),
polyethylene glycol(20)isostearyl ether (isosteareth-20);
Polyethylene glycol(13)cetyl ether (ceteth-13),
polyethylene glycol(14)cetyl ether (ceteth-14),
polyethylene glycol(15)cetyl ether (ceteth-15),
polyethylene glycol(16)cetyl ether (ceteth-16),
polyethylene glycol(17)cetyl ether (ceteth-17),
polyethylene glycol(18)cetyl ether (ceteth-18),
polyethylene glycol(19)cetyl ether (ceteth-19),
polyethylene glycol(20)cetyl ether (ceteth-20);
Polyethylene glycol(13)isocetyl ether (isoceteth-13),
polyethylene glycol(14)isocetyl ether (isoceteth-14),
polyethylene glycol(15)isocetyl ether (isoceteth-15),
polyethylene glycol(16)isocetyl ether (isoceteth-16),
polyethylene glycol(17)isocetyl ether (isoceteth-17),
polyethylene glycol(18)isocetyl ether (isoceteth-18),
polyethylene glycol(19)isocetyl ether (isoceteth-l9),
polyethylene glycol(20)isocetyl ether (isoceteth-20);
Polyethylene glycol(12)oleyl ether (oleth-12),
polyethylene glycol(13)oleyl ether (oleth-13),
polyethylene glycol(14)oleyl ether (oleth-14),
polyethylene glycol(15)oleyl ether (oleth-15);
Polyethylene glycol(12)lauryl ether (laureth-12),
polyethylene glycol(12)isolauryl ether (isolaureth-12);
Polyethylene glycol(13)cetylstearyl ether (ceteareth-13),
polyethylene glycol(14)cetylstearyl ether (ceteareth-14),
polyethylene glycol(15)cetylstearyl ether (ceteareth-15),
polyethylene glycol(16)cetylstearyl ether (ceteareth-16),
polyethylene glycol(17)cetylsteary lether (ceteareth-17),
polyethylene glycol(18)cetylstearyl ether (ceteareth-18),
polyethylene glycol(19)cetylstearyl ether (ceteareth-19),
polyethylene glycol(20)cetylstearyl ether (ceteareth-20).

It is also advantageous to select the fatty acid ethoxylates from the following group:
polyethylene glycol(20) stearate,
polyethylene glycol(21) stearate,
polyethylene glycol(22) stearate,
polyethylene glycol(23) stearate,
polyethylene glycol(24) stearate,
polyethylene glycol(25) stearate;
Polyethylene glycol(12) isostearate,
polyethylene glycol(13) isostearate,
polyethylene glycol(14) isostearate,
polyethylene glycol(15) isostearate,
polyethylene glycol(16) isostearate,
polyethylene glycol(17) isostearate,
polyethylene glycol(18) isostearate,
polyethylene glycol(19) isostearate,
polyethylene glycol(20) isostearate,
polyethylene glycol(21) isostearate,
polyethylene glycol(22) isostearate,
polyethylene glycol(23) isostearate,
polyethylene glycol(24) isostearate,
polyethylene glycol(25) isostearate;
Polyethylene glycol(12) oleate,
polyethylene glycol(13) oleate,
polyethylene glycol(14) oleate,
polyethylene glycol(15) oleate,
polyethylene glycol(16) oleate,
polyethylene glycol(17) oleate,
polyethylene glycol(18) oleate,
polyethylene glycol(19) oleate,
polyethylene glycol(20) oleate.

Sodiumlaureth-11-carboxylate may advantageously be used as ethoxylated alkyl ether carboxylic acid or its salts.

Sodium laureth 1-4 sulfate may advantageously be used as an alkyl ether sulfate.

Polyethylene glycol(30)cholesteryl ether may be advantageously used as ethoxylated cholesterol derivative. Polyethylene glycol(25)soyasterol has also proven successful.

Polyethylene glycol(60) evening primrose glycerides may advantageously be used as ethoxylated triglycerides.

It is also advantageous to select the fatty acid esters of polyethylene glycol glycerol from the group of polyethylene glycol(20)glyceryl laurate, polyethylene glycol(21)glyceryl laurate, polyethylene glycol(22)glyceryl laurate, polyethylene glycol(23)glyceryl laurate, polyethylene glycol(6)glyceryl caprate/caprinate, polyethylene glycol(20)glyceryl oleate, polyethylene glycol(20)glyceryl isostearate, polyethylene glycol(18)glyceryl oleate/cocoate.

It is likewise advantageous to select the sorbitan esters from the group of:
polyethylene glycol(20)sorbitan monolaurate,
polyethylene glycol(20)sorbitan monostearate,
polyethylene glycol(20)sorbitan monoisostearate,
polyethylene glycol(20)sorbitan monopalmitate,
polyethylene glycol(20)sorbitan monooleate.

It is possible to use as advantageous W/O emulsifiers: fatty alcohols having 8 to 30 carbon atoms, monoglycerol esters of saturated and/or unsaturated, branched and/or unbranched alkane carboxylic acids having a chain length from 8 to 24, in particular 12 to 18 carbon atoms, diglycerol esters of saturated and/or unsaturated, branched and/or unbranched alkane carboxylic acids having a chain length from 8 to 24, in particular 12 to 18 carbon atoms, monoglycerol ethers of saturated and/or unsaturated, branched and/or unbranched alcohols having a chain length from 8 to 24, in particular 12 to 18 carbon atoms, diglycerol ethers of saturated and/or unsaturated, branched and/or unbranched alcohols having a chain length from 8 to 24, in particular 12 to 18 carbon atoms, propylene glycol esters of saturated and/or unsaturated, branched and/or unbranched alkane carboxylic acids having a chain length from 8 to 24, in particular 12 to 18 carbon atoms, as well as sorbitan esters of saturated and/or unsaturated, branched and/or unbranched alkane carboxylic acids having a chain length from 8 to 24, in particular 12 to 18 carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2)stearyl ether (steareth-2), glyceryl monolaurate, glyceryl monocaprinate, glyceryl monocaprylate.

Especially preferred embodiments of the preparations according to the invention are characterized in that they contain one or more cyclodextrins and/or cyclodextrin derivatives. Cyclodextrins and/or cyclodextrin derivatives represent in accordance with the invention native cyclodextrins, alpha-, beta-, and gamma cyclodextrin, as well as the derivatives of these species, in particular all alpha-, beta-, gamma-cyclodextrins, which are fully or partially etherified at the hydroxyl groups, and/or esterified, and/or otherwise derivatized, such as alpha-cyclodextrin, beta-cyclodextrin, hydroxypropyl-beta-cyclodextrin, hydroxypropyl-gamma-cyclodextrin, partially methylated (random methyl-) beta-cyclodextrin, and/or gamma cyclodextrin.

The improvement of the solubility of sparingly soluble substances in the presence of cyclodextrins in an aqueous medium is described for individual substances. Of advantage may be both the inclusion compounds of a substance, also named guest, with a cyclodextrin species, wherein both 1:1 or 1:2 complexes as well as complexes with additional molar ratios (guest: cyclodextrin) are possible, and their physical mixture.

Cyclodextrins and their derivatives are able to form inclusion complexes because of their structure. They are suitable for a “molecular encapsulation” of active ingredients (for example, as protective covering of sensitive molecules in cosmetic and pharmaceutical formulations).

The cyclodextrins, or cyclodextrin-guest-inclusion complexes, or the cyclodextrin-substance mixtures can be easily incorporated into common cosmetic or dermatological formulations.

In accordance with the invention the cyclodextrin or cyclodextrins and/or their derivatives are advantageously used in a concentration of 0.0005% to 20% by weight, preferably in a concentration of 0.01% to 10% by weight, and very preferably in a concentration of 0.1% to 5% by weight based on the total weight of the preparation.

In accordance with the invention, the especially preferred cyclodextrins are gamma-cyclodextrin as well as hydroxypropyl-beta-cyclodextrin.

Likewise other forms of encapsulation of the 2,3-dibenzylbutyrolactone derivatives and -glycosides can be advantageously used for the purposes of the invention. Especially advantageous are, for example, the methods of encapsulating lipid particles as are disclosed in DE 199 45 203 A1, WO 94/20072, or WO 00/67728.

The invention is by no means limited to the referenced cosmetic or dermatological active ingredients, auxiliary agents, and additives.

In accordance with the invention, one or more 2,3-dibenzylbutyrolactone derivatives and/or their glycosides are used for producing a pharmaceutical preparation for the treatment and/or prophylaxis of skin aging symptoms.

In accordance with the invention, one or more 2,3-dibenzylbutyrolactone derivatives and/or their glycosides are furthermore used for producing a pharmaceutical preparation for the treatment and/or prophylaxis of inflammatory skin conditions.

Last but not least, it is in accordance with the invention to use one or more 2,3-dibenzylbutyrolactone derivatives and/or their glycosides for producing a pharmaceutical preparation for the treatment and/or prophylaxis of unclean skin and acne.

In accordance with the invention, one or more 2,3-dibenzylbutyrolactone derivatives and/or their glycosides are also used for producing a cosmetic for the treatment and/or prophylaxis of skin aging symptoms.

In accordance with the invention, one or more 2,3-dibenzylbutyrolactone derivatives and/or their glycosides are furthermore used for producing a cosmetic for the treatment and/or prophylaxis of inflammatory skin conditions.

Last but not least, it is in accordance with the invention to use one or more 2,3-dibenzylbutyrolactone derivatives and/or their glycosides for producing a cosmetic for the treatment and/or prophylaxis of unclean skin and acne.

The prophylaxis and the cosmetic or dermatological treatment respectively with the 2,3-dibenzylbutyrolactone derivatives and/or their glycosides as used in accordance with the invention, or with the cosmetic or topical dermatological preparations with an active content of the 2,3-dibenzylbutyrolactone derivatives and/or their glycosides as used in accordance with the invention, occurs in a customary manner such that one applies to the affected skin parts the 2,3-dibenzylbutyrolactone derivatives and/or their glycosides as used in accordance with the invention, or the cosmetic or topical dermatological preparations with an active content of the 2,3-dibenzylbutyrolactone derivatives and/or their glycosides as used in accordance with the invention.

Besides health and care of the skin, a further branch of cosmetics also includes hair care. Surprisingly, the preparations of the invention are suited for the treatment and care of the skin appendages, i.e., nails, sweat glands, sebaceous glands, hair follicles, and in particular hair. In accordance with the invention, the treatment and care of oily hair and/or dandruff are especially advantageous, since these appearances exhibit a certain relationship with unclean skin (increased sebum production), or with dry inflammatory skin. In this connection, the preparations can be advantageously used in the form of shampoos, conditioners, rinses, and cures. With that, the invention also provides for using one or more 2,3-dibenzylbutyrolactone derivatives and/or their glycosides for the production of a pharmaceutical and/or cosmetic for the treatment and care of the skin appendages.

The following examples are intended to illustrate the present invention, without limiting it. Unless otherwise specified, all specified quantities, constituents, and percentages are based on the weight, total quantity, or total weight of the preparations.

### EXAMPLES

1. O/W Cream

| | Examples | | | | |
|---|---|---|---|---|---|
| | 1 | 2 | 3 | 4 | 5 |
| Glyceryl stearate citrate | | | 2.00 | | 2.00 |
| Glyceryl stearate, | 4.00 | 3.00 | | | |
| self-emulsifying | | | | | |
| PEG-40-stearate | 1.00 | | | | |
| Polyglyceryl-3- | | | | 3.00 | |
| methylglucose-distearate | | | | | |
| Sorbitan stearate | | | | | 2.00 |
| Stearic acid | | 1.00 | | | |
| Polyoxyethylene (20)- | | | | | |
| cetylstearyl ether | | | | | |
| Stearyl alcohol | | | 5.00 | | |
| Cetyl alcohol | 3.00 | 2.00 | | 3.00 | |
| Cetylstearyl alcohol | | | | | 2.00 |
| C12-15 alkylbenzoate | | | | | |
| Caprylic-/capric | 5.00 | 3.00 | 4.00 | 3.00 | 3.00 |
| triglyceride | | | | | |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidene | | | 1.00 | | |
| camphor | | | | | |
| 1-(4-tert-butylphenyl)-3- | | | 0.50 | | |
| (4-methoxyphenyl)-1,3- | | | | | |
| propane-dione | | | | | |
| 2,3-dibenzyl- | 0.20 | 0.50 | 0.10 | 1.00 | 0.30 |
| butyrolactone | | | | | |
| derivatives and/or | | | | | |
| their glycosides | | | | | |
| Tocopherol | 0.1 | | | | 0.20 |
| Biotin | | | 0.05 | | |
| Ethylene | 0.1 | | 0.10 | 0.1 | |
| diaminetetraacidic | | | | | |
| acid trisodium | | | | | |
| Preservative | q.s. | q.s. | q.s. | q.s. | q.s. |
| Xanthan gum | | | | | |
| Polyacrylic acid | 3.00 | 0.1 | | 0.1 | 0.1 |
| Sodium hydroxide 45% | q.s | q.s. | q.s. | q.s. | q.s. |
| Glycerol | 5.00 | 3.00 | 4.00 | 3.00 | 3.00 |
| Butylene glycol | | 3.00 | | | |
| Perfume | q.s | q.s. | q.s. | q.s. | q.s. |
| Water | ad 100 | ad 100 | ad 100 | ad 100 | ad 100 |

2. O/W Cream

| | Examples | | | | |
|---|---|---|---|---|---|
| | 6 | 7 | 8 | 9 | 10 |
| Glyceryl stearate citrate | | 2.00 | 2.00 | | |
| Glyceryl stearate, | 5.00 | | | | |
| self-emulsifying | | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic-/capric | 2.00 | | | | |
| triglyceride | | | | | |
| Octyldodecanol | 2.00 | 2.00 | | 4.00 | 6.00 |
| Dicaprylylether | | | | | |
| Paraffinum liquidum | | 4.00 | 2.00 | | |
| Cyclic dimethyl | | | | 0.50 | 2.00 |
| polysiloxane | | | | | |
| Dimethicone | 2.00 | | | | |
| polydimethylsiloxane | | | | | |
| Titanium dioxide | 2.00 | | | | |
| 4-methylbenzylidene | 1.00 | | | | 1.00 |
| camphor | | | | | |
| 1-(4-tert-butylphenyl)-3- | 0.50 | | | | 0.50 |
| (4-methoxyphenyl)- | | | | | |
| 1,3-propane-dione | | | | | |
| 2,3-dibenzyl- | 0.20 | 0.70 | 0.25 | 1.00 | 0.40 |
| butyrolactone | | | | | |
| derivatives and/or | | | | | |
| their glycosides | | | | | |
| Tocopherol | | | | | 0.05 |
| Ethylene | | | 0.20 | | 0.20 |
| diaminetetraacetic | | | | | |
| acid trisodium | | | | | |
| Preservative | q.s | q.s. | q.s. | q.s. | q.s. |
| Xanthan gum | | | 0.20 | | |
| Polyacrylic acid | 0.15 | 0.1 | | 0.05 | 0.05 |
| Sodium hydroxide 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

3. W/O-Emulsions

| | Examples | | | | |
|---|---|---|---|---|---|
| | 1 | 2 | 3 | 4 | 5 |
| Cetyldimethicone copolyol | | 2.50 | | 4.00 | |
| Polyglyceryl-2-dipolyhydroxy stearate | 5.00 | | | | 4.50 |
| PEG-30-dipolyhydroxy stearate | | | 5.00 | | |
| 2-ethylhexyl methoxy cinnamate | | 8.00 | | 5.00 | 4.00 |
| 2,4-bis-(4-(2-ethyl-hexyloxy-)2-hydroxyl)- | 2.00 | 2.50 | | 2.00 | 2.50 |
| phenyl)-6-(4-methoxyphenyl)-(1,3,5)-triazine | | | | | |
| 1-(4-tert-butylphenyl)-3-(4-methoxyphenyl)- | | | 2.00 | 1.00 | |
| 1,3-propane dione | | | | | |
| Diethylhexyl butamidotriazone | 3.00 | 1.00 | | | 3.00 |
| Ethylhexyl triazone | | | 3.00 | 4.00 | |
| 4-methylbenzylidene camphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4-bis-(monosodium, 2- | 1.00 | 2.00 | 0.50 | | |
| benzimidazyl-5,7-disulfonic acid) | | | | | |
| Phenylbenzimidazol sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C12-15 alkyl benzoate | | | | 9.00 | |
| Dicaprylyl ether | 10.00 | | | | 7.00 |
| Butyleneglycol-dicaprylate/-dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethyl polysiloxane | 2.00 | 25.00 | | | 2.00 |
| Shea butter | | | 3.00 | | |
| PVP hexadecane copolymer | 0.50 | | | 0.50 | 1.00 |
| Octoxyglycerin | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| 2,3-dibenzylbutyrolactone derivatives and/or | 0.10 | 0.60 | 1.00 | 1.00 | 0.8 |
| their glycosides | | | | | |
| Preservative | q.s | q.s. | q.s | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

4. W/O Emulsions

Examples

| | 6 | 7 |
|---|---|---|
| Polyglyceryl-2-dipolyhydroxy stearate | 4.00 | 5.00 |
| PEG-30-dipolyhydroxy stearate | | |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| 1-(4-tert-butylphenyl)-3-(4-methoxyphenyl)- | 0.50 | 1.50 |
| 1,3-propane-dione | | |
| 4-Methylbenzylidene camphor | | 300 |
| Butylene glycol dicaprylate/-dicaprate | 4.00 | 5.00 |
| Shea butter | | 0.50 |
| Butylene glycol | | 6.00 |
| Octoxyglycerin | | 3.00 |
| Glycerol | 5.00 | |
| Tocopherol acetate | 0.50 | 1.00 |
| 2,3-dibenzylbutyrolactone derivatives and/or | 0.20 | 0.45 |
| their glycosides | | |
| Ethylene diamintetraacetic acid | 0.20 | 0.20 |
| Preservative | q.s. | q.s. |
| Ethanol | | 3.00 |
| Perfume | q.s. | q.s. |
| Water | ad. 100 | ad. 100 |

5. Hydrodispersions

| | Examples | | | | |
|---|---|---|---|---|---|
| | 1 | 2 | 3 | 4 | 5 |
| Polyoxyethylene(20)cetylstearyl ether | 1.00 | | | 0.5 | |
| Cetylalcohol | | | 1.00 | | |
| Sodium polyacrylate | | 0.20 | | 0.30 | |
| Acrylate/C10-30-alkyl-acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxy cinnamate | | | | 5.00 | 8.00 |
| 2,4-bis-(4-(2-ethyl-hexyloxy-)2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-butylphenyl)-3-(4- | 1.00 | | 2.00 | | |
| methoxyphenyl)-1,3-propane-dione | | | | | |
| Diethylhexyl butamidotriazone | | 2.00 | | 2.00 | 1.00 |
| Ethylhexyl triazone | 4.00 | | 3.00 | 4.00 | |
| 4-methylbenzylidene camphor | | | | | 2.00 |
| Octocrylene | | 4.00 | 4.00 | | 2.50 |
| Phenylene-1,4-bis-(monosodium, 2- | 1.00 | | 0.50 | | 2.00 |
| benzimidazyl-5,7-disulfonic acid | | | | | |
| Phenylbenzimidazol sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/-dicaprate | 4.00 | | 2.00 | 6.00 | |
| Dicaprylyl carbonate | | 2.00 | 6.00 | | |
| Dimethicone polydimethyl siloxane | | 0.50 | 1.00 | | |
| Phenyimethyl polysiloxane | 2.00 | | | 0.50 | 2.00 |
| Shea butter | | 2.00 | | | |
| PVP hexadecane copolymer | 0.50 | | | 0.50 | 1.00 |
| Octoxyglycerin | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | |
| 2,3-dibenzylbutyrolactone derivatives | 0.15 | 0.60 | 1.00 | 1.00 | 0.80 |
| and/or their glycosides | | | | | |
| Preservative | q.s. | q.s. | q.s. | q.s., | q.s. |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

6. Gel Cream

| | |
|---|---|
| Acrylate/C10-30 alkylacrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethyl polysiloxane | 5.00 |
| Dimeticone polydimethyl siloxane | 1.00 |
| 2,3-dibenzylbutyrolactone derivatives | 0.05 |
| and/or their glycosides | |
| Glycerol | 3.00 |
| Sodium hydroxide | q.s. |
| Preservative | q.s. |
| Perfume | q.s. |
| Water | ad 100 |
| pH-value adjusted to 6.0 | |

7. W/O Cream

| | |
|---|---|
| Polyglyceryl-diisostearate | 3.50 |
| Glycerol | 3.00 |
| Polyglyceryl-2-dipolyhydroxy stearate | 3.50 |
| 2,3-dibenzylbutyrolactone derivatives | 0.60 |
| and/or their glycosides | |
| Preservative | q.s. |
| Perfume | q.s. |
| Water | ad 100 |
| Magnesium sulfate | 0.6 |
| Isopropyl stearate | 2.0 |
| Caprylyl ether | 8.0 |
| Cetearyl isononanoate | 6.0 |

8. W/O/W Cream

| | |
|---|---|
| Glyceryl stearate | 3.00 |
| PEG-100 stearate | 0.75 |
| Behenyl alcohol | 2.00 |
| Caprylic-/capric-trigiyceride | 8.0 |
| Octyldodecanol | 5.00 |
| C12-15 alkyl benzoate | 3.00 |
| 2,3-dibenzylbutyrolactone derivatives | 0.50 |
| and/or their glycosides | |
| Magnesium sulfate (MgSO₄) | 0.80 |
| Ethylene diaminetetraacetic acid | 0.10 |
| Preservative | q.s. |
| Perfume | q.s. |
| Water | ad 100 |
| pH-value adjusted to 6.0 | |

9. Shower Bath

| | |
|---|---|
| Sodium laureth sulfate | 33.00 |
| Potassium-cocoyl hydrolyzed collagen (30%) | 11.00 |
| Cocoamphodiacetate (30%) | 5.00 |
| PEG-7 gyceryl cocoate | 2.00 |
| Cocamide MEA | 1.00 |
| Sodium chloride | 0.50 |
| 2,3-dibenzyibutyrolactone derivatives and/or | 0.05 |
| their glycosides | |
| Citric acid | 0.02 |
| Preservatives, dyes, perfume | q.s. |
| Water | ad 100 |

10. Hair Cure

| | |
|---|---|
| Hydroxypropylmethyl cellulose | 0.50 |
| Cetrimonium bromide | 1.00 |
| Glycerol | 3.00 |
| Cetearyl alcohol | 2.50 |
| Benzophenone-4 | 0.4 |
| Glyceryl stearate | 2.00 |
| 2,3-dibenzyibutyrolactone derivatives and/or | 0.1 |
| their glycosides | |
| Preservative, perfume, pH adjustment | q.s. |
| Water | ad 100 |
| The pH-value is adjusted to 3.5. | |

11. Hair Rinse

| | |
|---|---|
| Behentrimonium chloride | 1.00 |
| Glycerin | 3.00 |
| Benzophenone-4 | 0.25 |
| Hydroxyethyl cellulose | 0.20 |
| Cetearyl alcohol | 3.00 |
| 2,3-dibenzylbutyrolactone derivatives and/or | 0.2 |
| their glycosides | |
| Folic acid | 0.80 |
| Preservative, perfume, pH-adjustment | q.s. |
| Water | ad 100 |
| The pH-value is adjusted to 6. | |

12 Conditioner Shampoo with Pearly Luster

| | Examples | | |
|---|---|---|---|
| | 1 | 2 | 3 |
| Polyquaternium-10 | 0.5 | 0.5 | 0.5 |
| Sodium laureth sulfate | 9.0 | 9.0 | 9.0 |
| Benzophenone-3 | | 0.5 | |
| Benzophenone-4 | | | 0.4 |
| Cocoamidopropyl betaine | 2.5 | 2.5 | 2.5 |
| Pearlescent agent | 2.0 | 2.0 | 2.0 |
| 2,3-dibenzylbutyrolactone derivatives | 0.06 | 0.15 | 0.01 |
| and/or their glycosides | | | |
| Disodium EDTA | 0.1 | 0.2 | 0.15 |
| Preservative, perfume, thickener, pH- | q.s. | q.s. | q.s. |
| adjustment, and solubilizer | | | |
| Water, VES (fully desalted) | ad 100 | ad 100 | ad 100 |
| The pH-value is adjusted to 6. | | | |

13. Clear Conditioner Shampoo

| | Examples | | |
|---|---|---|---|
| | 1 | 2 | 3 |
| Polyquaternium-10 | 0.5 | 0.5 | 0.5 |
| Benzophenone-4 | | 0.4 | |
| 2-Ethylhexyl methoxy cinnamate | | | 0.2 |
| Sodium laureth sulfate | 9.0 | 9.0 | 9.0 |
| Cocoamidopropyl betaine | 2.5 | 2.5 | 2.5 |
| 2,3-dibenzylbutyrolactone derivatives | 0.02 | 0.05 | 0.05 |
| and/or their glycosides | | | |
| Iminodisuccinic acid, sodium salt | 0.2 | 0.3 | 0.8 |
| Preservative, perfume, thickener, pH- | q.s. | q.s. | q.s. |
| adjustment, and solubilizer | | | |
| Water, VES (fully desalted) | ad 100 | ad 100 | ad 100 |
| The pH-value is adjusted to 6. | | | |

14. Clear Light Shampoo with Volume Effect

| | Examples | | |
|---|---|---|---|
| | 1 | 2 | 3 |
| Sodium laureth sulfate | 10.0 | 10.0 | 10.0 |
| Cocoamidopropyl betaine | 2.5 | 2.5 | 2.5 |
| 2,3-dibenzylbutyrolactone derivatives | 0.5 | 0.6 | 0.3 |
| and/or their glycosides | | | |
| Disodium EDTA | 0.2 | 0.15 | 0.7 |
| Preservative, perfume, thickener, pH- | q.s. | q.s, | q.s. |
| adjustment, solubilizer | | | |
| Water, VES (fully desalted) | ad 100 | ad 100 | ad 100 |
| The pH-value is adjusted to 5.5.. | | | |

1. A cosmetic or dermatological preparation comprising at least one compound selected from the group consisting of 2,3-dibenzylbutyrolactone, 2,3-dibenzylbutvrolactone derivatives, glycosides of 2.3-dibenzylbutyrolactone, and glycosides of 2,3-dibenzylbutyrolactone derivatives.

2. The cosmetic or dermatological preparation as claimed in claim 1, wherein the preparation is present in the form of an emulsion.

3. The cosmetic or dermatological preparation as claimed in claim 1, wherein the at least one compound is present in a concentration of 0.001% to 10% by weight, based on the total weight of the preparation.

4. The cosmetic or dermatological preparation as claimed in claim 1, wherein the at least one compound includes at least one plant extract.

5. The cosmetic or dermatological preparation as claimed in claim 4, wherein the at least one plant extract includes at least one extract selected from the group consisting of Arctium lappa L. (greater burdock) extracts, Steganotaenia araliacea (carrot tree) extracts, and mixtures thereof.

6. The cosmetic or dermatological preparation as claimed in claim 1, wherein the at least one compound includes at least one compound selected from the group consisting of arctiin, arctigenin, prestegane B, matairesinol, tracheloside, and trachelogenin.

7. A method for treating or caring for the skin or hair comprising applying to the skin or hair a cosmetic or dermatological preparation comprising at least one compound selected from the group consisting of 2,3-dibenzylbutvrolactone, 2,3-dibenzylbutyrolactone derivatives, glycosides of 2,3-dibenzylbutvrolactone, and glycosides of 2,3-dibenzylbutvrolactone derivatives.

8. The method as claimed in claim 7, wherein the cosmetic or dermatological preparation is applied to the skin for the treatment or prophylaxis of inflammatory skin conditions.

9. The method as claimed in claim 7, wherein the cosmetic or dermatolorical preparation is applied to the skin for the treatment or prophylaxis of unclean skin and acne.

10. The method as claimed in claim 7, wherein the cosmetic or dermatological preparation is applied to the skin or hair for the treatment and care of hair and skin appendages.

11. The method as claimed in claim 7, wherein the cosmetic or dermatological preparation is applied to the skin for the treatment or prophylaxis of skin aging symptoms.

12. The method as claimed in claim 7, wherein the at least one compound includes at least one compound selected from the group consisting of arctiin, arctigenin, prestegane B, matairesinol, tracheloside,-and trachelogenin.

13. The method as claimed in claim 7, wherein the at least one compound includes at least one plant extract selected from the group consisting of Arctium lappa L. (greater burdock) extracts, Steganotaenia araliacea (carrot tree) extracts, and mixtures thereof.

14. The cosmetic or dermatological preparation of claim 1, further comprising at least one compound selected from the group consisting of cosmetic or dermatological active ingredients, auxiliary agents, and additives.

15. The cosmetic or dermatological preparation of claim 3, wherein the at least one compound is present in a concentration of 0.05% to 5% by weight based on the total weight of the preparation.

16. The cosmetic or dermatological preparation of claim 15, wherein the at least one compound is present in a concentration of 0.01% to 2% by weight based on the total weight of the preparation.
